(19) United States
(12) Patent Application Publication
(10) Pub. No.: US 2014/0294755 A1
(43) Pub. Date: Oct. 2, 2014

(54) Materials and Methods Relating to Stem Cell Mobilization by Multi-Pegylated Granulocyte Colony Stimulating Factor

(71) Applicants: Ravi Ali, Newbury Park, CA (US); Geoff Hill, Brisbane (AU); Jeffrey Martin Hogan, Thousand Oaks, CA (US); Pamela Sue McGarva, Hingham, MA (US); Graham Molineux, Moorpark, CA (US); Ali Siahpush, Bellevue, WA (US)

(72) Inventors: Ravi Ali, Newbury Park, CA (US); Geoff Hill, Brisbane (AU); Jeffrey Martin Hogan, Thousand Oaks, CA (US); Pamela Sue McGarva, Hingham, MA (US); Graham Molineux, Moorpark, CA (US); Ali Siahpush, Bellevue, WA (US)

(73) Assignee: AMGEN INC., Thousand Oaks, CA (US)

(21) Appl. No.: 14/227,325

(22) Filed: Mar. 27, 2014

Related U.S. Application Data
(63) Continuation of application No. 13/994,594.
(60) Provisional application No. 61/110,224, filed on Oct. 31, 2008.

(51) Int. Cl. A61K 47/48; A61K 35/28; A61K 45/06; A61K 38/19

(52) U.S. Cl. CPC A61K 47/48215; A61K 38/193; A61K 35/28; A61K 45/06
USPC 424/85.1; 530/351; 435/325

(57) ABSTRACT

The present invention relates to the use of multi-PEGylated granulocyte colony stimulating factor (G-CSF) preparations to mobilize hematopoietic stem cells.

### FIELD OF THE INVENTION

The present invention relates to the use of multi-PEGylated granulocyte colony stimulating factor (G-CSF) polypeptide to mobilize hematopoietic stem cells.

### BACKGROUND OF THE INVENTION

Stem cell transplantation (SCT) is one procedure used to treat people suffering from diseases of the blood or bone marrow, as well as certain types of cancer. Pluripotent stem cells are progenitor cells that are able to turn or “differentiate” into many types of cells including blood cells. When transplanted into a recipient patient, the cells can populate the patient's bone marrow and produce new blood cells. Many recipients of SCTs are multiple myeloma and leukemia patients who would not benefit from prolonged treatment with, or are already resistant to, chemotherapy or total body irradiation. Other candidates for SCTs include pediatric cases where the patient has an inborn defect such as severe combined immunodeficiency or congenital neutropenia with defective stem cells, and also children or adults with aplastic anemia who have lost their stem cells after birth. Other conditions treated with SCTs include sickle-cell disease, myelodysplastic syndrome, neuroblastoma, lymphoma, Ewing's Sarcoma, Desmoplastic small round cell tumor and Hodgkin's disease.

Bone marrow transplantation was a precursor to SCT. After the discovery and development of growth factors such as G-CSF, most hematopoietic SCT procedures are now performed using stem cells collected from the peripheral blood, rather than bone marrow itself. The administration of G-CSF and stem cell factor has been shown to mobilize pluripotent stem cells from the bone marrow and greatly increase their number in the peripheral circulation [Orlic et al., Proc. Natl. Acad. Sci. USA, 98:10344-10349 (2001)]. Hematopoietic stem cells are collected from the blood through a process known as apheresis. A donor's blood is withdrawn through a sterile needle and passed through a machine that removes stem cells. The red blood cells are returned to the donor. The peripheral stem cell yield is boosted with daily subcutaneous injections of G-CSF given for a period of days before apheresis.

Autologous SCT involves isolation of stem cells from a patient and storage of the harvested cells in a freezer. The patient is then treated with high-dose chemotherapy, with or without radiotherapy in the form of total body irradiation, to eradicate the patient's malignant blood cell population. The patient's own stored stem cells are then reintroduced. After entering the bloodstream, the transplanted cells travel to the bone marrow, where they begin to produce new white blood cells, red blood cells, and platelets in a process known as “engraftment.” Engraftment usually occurs within about two to four weeks after transplantation, and is monitored by checking blood counts on a frequent basis. Complete recovery of immune function takes much longer, up to several months for autologous transplant recipients and one to two years for patients receiving allogeneic transplants. Allogeneic SCT involves two people: a donor and a patient recipient. In allogeneic SCT, while stem cell donors are selected to have a tissue type that is the best match possible for the patient, the patient must take immunosuppressive medications to mitigate graft-versus-host disease (GVHD).

GVHD is an inflammatory disease that is unique to allogeneic transplantation. It is an attack of the donor immune cells on the recipient patient's body tissues. Acute GVHD typically occurs in the first 100 days after SCT and may involve the skin, gastrointestinal tract and liver, and is often fatal. High-dose corticosteroids such as prednisone are a standard treatment, but this immunosuppressive treatment often leads to deadly infections. Chronic GVHD may also develop after allogeneic transplant (more than 100 days after transplant). It is the major source of late treatment-related complications, although it less often results in death. In addition to inflammation, chronic GVHD may lead to the development of cutaneous and hepatic fibrosis. It may cause functional disability and require prolonged immunosuppressive therapy. GVHD is usually mediated by donor T cells.

T cells from donors treated with G-CSF have a reduced capacity to induce GVHD on a per cell basis relative to those from control-treated donors [Pan et al., Blood, 86: 4422-4429 (1995)] and G-CSF may also reduce GVHD through effects on dendritic cells, monocytes and natural killer cells [reviewed in Morris et al., Blood, 107: 3430-3435 (2006)]. Moreover,—PEGylated-G-CSF is superior to standard G-CSF for the prevention of GVHD, whilst paradoxically improving GVL via iNKT-dependent effects. See, Morris et al., J. Clin. Invest., 115: 3093-3103 (2005) and Morris et al., Blood, 103: 3573-3581 (2004). Phase I clinical studies in normal donors have demonstrated that 12 mg of mono-PEGylated G-CSF SD/01 (100-200 μg/kg) results in robust stem cell mobilization with an acceptable side effect profile. See Hill et al., Biol. Blood Marrow Transplant, 12: 603-607 (2006).

In contrast to GVHD, there is a beneficial aspect of the Graft-versus-Host phenomenon that is known as the “graft versus tumor” (GVT) or “graft versus leukemia” (GVL) effect. For example, SCT patients with either acute or, in particular, chronic GVHD after allogeneic transplant tend to have a lower risk of cancer relapse. This is due to a therapeutic immune reaction of the grafted donor lymphocytes, including natural killer (NK) cells, against any diseased bone marrow of the recipient. This lower rate of relapse accounts for the increased success rate of allogeneic transplants compared to transplants from identical twins, and indicates that allogeneic SCT is a form of immunotherapy. GVT is the major benefit of transplants which do not employ the highest immunosuppressive regimens.

There remains a need in the art for improved methods and materials for SCT that minimize GVHD while maximizing GVT effects.

### SUMMARY OF THE INVENTION

The present invention provides methods and materials for mobilizing hematopoietic stem cells. The use of multi-PEGylated G-CSF preparations of the invention to mobilize hematopoietic stem cells results in greater levels of myeloid expansion in a treated donor. Moreover, after transplant of the donor cells, enhanced CTL function and improved GVT effects are seen in a transplant recipient. Improved immunomodulatory and/or anti-tumor effects of cells arising from stimulation with multi-PEGylated G-CSF preparations of the invention may also be seen in other patients (i.e., patients not receiving a SCT) when those patients are treated with the preparations.

### G-CSF Preparations of the Invention

In one aspect, the invention provides multi-PEGylated G-CSF preparations. Such G-CSF preparations comprise G-CSF polypeptides (e.g., Filgrastim), each with polyethylene glycol (PEG) moieties attached at two or more sites. Numerous PEG molecules are known in the art. Different multi-PEGylated G-CSF preparations of the invention may comprise PEG moieties of different molecular weights. One preparation may comprise 20 kDa PEG moieties while another preparation may comprise 1 kDa PEG moieties. PEG moieties, including but not limited to. PEG moieties ranging from about 1 KDa to about 20 kDa are contemplated by the invention.

One multi-PEGylated G-CSF preparation of the invention is named “SD/03.” SD/03 comprises Filgastrim polypeptides, each with PEG moieties (20 kDa) attached at two or more sites. Example 1 describes a method of making an SD/03 preparation.

Multi-PEGylated G-CSF preparations of the invention may be made by attaching PEG-aldehyde moieties to granulocyte colony stimulating polypeptide by reductive alkylation in the presence of a reducing agent such as sodium cyanoborohydride. The reductive alkylation reaction may be carried out for about 8 to about 24 hours. It may be conducted out at about ambient temperature. It may carried out at a pH from about pH 6 to about pH 8.5. The multi-PEGylated polypeptide is then separated from unreacted and mono-PEGylated polypeptide. In one embodiment, 20 kDa PEG-aldehyde moieties are attached to Filgastrim G-SCF polypeptide by a reductive alkylation reaction in which the reaction is carried out for 8 to 24 hours at ambient temperature and at a pH from pH 6 to pH 8.5 in the presence of sodium cyanoborohydride. The multi-PEGylated polypeptide is then separated from unreacted and mono-PEGylated polypeptide.

Human G-CSF polypeptides can be obtained and purified from a number of sources. Natural human G-CSF polypeptides can be isolated from the supernatants of cultured human tumor cell lines. The development of recombinant DNA technology has enabled the production of commercial scale quantities of G-CSF polypeptides in glycosylated form as a product of eukaryotic host cell expression, and of G-CSF polypeptides in non-glycosylated form as a product of prokaryotic host cell expression. See, for example, U.S. Pat. No. 4,810,643 (Souza) incorporated herein by reference.

The term “G-CSF polypeptide” or “G-CSF” as used herein is defined as naturally occurring human and heterologous species G-CSF, recombinantly produced G-CSF that is an expression product consisting of either 174 or 177 amino acids, or fragments, analogs, variants, or derivatives thereof as reported, for example in Kuga et al., Biochem. Biophys. Res. Comm. 159: 103-111 (1989); Lu et al., Arch. Biochem. Biophys. 268: 81-92 (1989); U.S. Pat. Nos. 4,810,643, 4,904,584, 5,104,651, 5,194,592, 5,214,132, 5,218,092, 5,362,853, 5,416,195, 5,606,024, 5,681,720, 5,714,581, 5,773,581, 5,795,968, 5,824,778, 5,824,784, 5,939,280, 5,994,518, 6,017,876, 6,027,720, 6,166,183, and 6,261,550; U.S. Pat. Appl. No. U.S. 20030064922; EP 0 335423; EP 0 272703; EP 0 459630; EP 0 256843; EP 0 243153; WO 9102874; Australian Application document Nos. AU-A-1094892 and AU-A-7638091. G-CSF analogs and G-CSF PEGylated analogs having G-CSF bioactivity are described in Nissen WO 03006501 and Osslund U.S. Pat. Nos. 5,581,476; 5,790,421; and 7,381,804. Also included are chemically modified G-CSFs, see, e.g., those reported in WO 9012874, EP 0 401384 and EP 0335423. See also, WO 03006501; WO 03030821; WO 0151510; WO 9611953; WO 9521629; WO 9420069; WO 9315211; WO 9305169; JP 04164098; WO 9206116; WO 9204455; EP 0 473268; EP 0 456200; WO 9111520; WO 9105798; WO 9006952; WO 8910932; WO 8905824; WO 9118911; and EP 0 370205. Also encompassed herein are all forms of G-CSF, such as Albugranin™, Neulasta™®, Neupogen®, Lenograstim, Nartogastim, Tevagrastim, Ratiograstim, Biograstim, Filgrastim, Filgrastim ratiopharm, Maxy-G34, GlycoPEG-G-CSF and Granocyte®. G-CSF derivatives include molecules modified by the addition of amino acids, including fusion proteins (procedures for which are well-known in the art). Such derivatization may occur singularly at the N- or C-terminus or there may be multiple sites of derivatization. Substitution of one or more amino acids with lysine may provide additional sites for derivatization. (See U.S. Pat. No. 5,824,784 and U.S. Pat. No. 5,824,778, incorporated by reference herein). G-CSF polypeptide may be generated by recombinant means or by automated peptide synthesis.

Pharmaceutical compositions comprising effective amounts of a G-CSF preparation of the invention together with pharmaceutically acceptable diluents, preservatives, solubilizers, emulsifiers, adjuvants and/or carriers are also provided. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimersol, benzyl alcohol), and bulking substances (e.g., lactose, mannitol); incorporation of the material into particulate preparations of polymeric compounds, such as polylactic acid, polyglycolic acid, etc., or in association with liposomes or micelles. Such compositions will influence the physical state, stability, rate of in vivo release, and rate of in vivo clearance of the G-CSF. See, e.g., Remington's Pharmaceutical Sciences, 18th Ed. (1990) Mack Publishing Co., Easton, Pa., pages 1435-1712, which are herein incorporated by reference.

### Administration of G-CSF Compositions of the Invention

Multi-PEGylated G-CSF preparations of the invention are formulated into appropriate pharmaceutical compositions as described above and administered to one or more sites within a donor in a therapeutically effective amount. By “effective amount” the present invention refers to that amount of multi-PEGylated G-CSF preparation sufficient to mobilize hematopoietic stem cells in methods of the invention.

The pharmaceutical compositions of the invention may be administered by any conventional method, e.g., by subcutaneous, intravenous or intradermal delivery This treatment may consist of a single dose followed by apheresis timed to maximize recovery of the cells to be transplanted. Giving a plurality of doses over a period of time (for example, one dose a day for five days) is also contemplated.

In addition to therapies based solely on the delivery of multi-PEGylated G-CSF preparations of the present invention, combination treatment is specifically contemplated. Multi-PEGylated G-CSF preparations of the invention may be used in conjunction with at least one other therapeutic agent (second therapeutic agent) including, but not limited to, stem cell factor, chemokine antagonists (e.g., AMD3100) or VCAM inhibitors. In some embodiments, second therapeutic agents such as stem cell factor promote mobilization of hematopoietic stem cells to the circulation, heart, bone marrow, and other organs.

The term “stem cell factor” or “SCF” as used herein refers to naturally-occurring SCF (e.g. natural human-SCF) as well as non-natural)y occurring (i.e., different from naturally occurring) polypeptides having amino acid sequences and glycosylation sufficiently duplicative of that of naturally-occurring stem cell factor to allow possession of a hematopoietic biological activity of naturally-occurring stem cell factor. The term “SCF” as used herein is also defined as recombinantly produced SCF, or fragments, analogs, variants, or derivatives thereof as reported, for example in U.S. Pat. Nos. 6,204,363, 6,207,417, 6,207,454, 6,207,802, 6,218,148, and 6,248,319. Stem cell factor has the ability to stimulate growth of early hematopoietic progenitors which are capable of maturing to erythroid, megakaryocyte, granulocyte, lymphocyte, and macrophage cells. SCF treatment of mammals results in absolute increases in hematopoietic cells of both myeloid and lymphoid lineages. One of the hallmark characteristics of stem cells is their ability to differentiate into both myeloid and lymphoid cells [Weissman, Science 241:58-62 (1988)].

It is also contemplated that one or more second therapeutic agents may be EPO, MGDF, SCF, GM-CSF, M-CSF, CSF-1, IL-1, IL-2, IL-3, IL-4, IL-5, IL-6, IL-7, IL-8, IL-9, IL-10, IL-11, IL-12, IL-18 (or various other interleukins), IGF-1, LIF, interferon (such as a, β, gamma or consensus), neurotrophic factors (such as BDNF, NT-3, CTNF or noggin), other multi-potent growth factors (such as, to the extent these are demonstrated to be such multi-potent growth factors, flt-3/flk-2 ligand, stem cell proliferation factor, and totipotent stem cell factor), fibroblast growth factors (such as FGF) or human growth hormone, chemokine inhibitors (such as AMD3100), or VCAM inhibitors as well as analogs, fusion molecules or derivatives thereof. For example, G-CSF in combination with SCF has been found to mobilize peripheral blood progenitor cells in vivo. Ex vivo, for example, G-CSF in combination with SCF. IL-3 and IL-6 has been found useful for expansion of peripheral blood cells.

In combination treatment, compositions are provided in a combined amount effective to produce the desired therapeutic outcome in the mobilization of c-Kit+ hematopoietic stem cells. This process may involve contacting the cells with the G-CSF composition and the second agent(s) at the same time. This may be achieved by administering a single composition or pharmacological formulation that includes both agents, or by administering two distinct compositions or formulations, at the same time, wherein one composition includes the human G-CSF composition and the other includes the second therapeutic agent.

Alternatively, the treatment with a multi-PEGylated G-CSF composition of the invention may precede or follow the treatment with the second agent(s) by intervals ranging from minutes to weeks. In embodiments where the second therapeutic agent and the human G-CSF composition are administered separately, one would generally ensure that a significant period of time did not expire between the times of each delivery, such that the second agent and the G-CSF composition would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Generally, an effective amount of G-CSF (calculating the mass of protein alone without chemical modification), or derivatives thereof, will be determined by the age, weight and condition of the donor. See, Remington's Pharmaceutical Sciences, supra, pages 697-773, herein incorporated by reference. Typically, a dosage of between about 0.001 μg/kg body weight/day to about 1000 μg/kg body weight/day may be used, but more or less as a skilled practitioner will recognize may be used. Dosages in an adult human may be approximately 100 to 500 μg/kg, 100 to 300 μg/kg, 10 to 500 μg/kg, 5 to 20 μg/kg, or 5 to 10 μg/kg. Administration of about 6 to about 12 mg in a single shot is also contemplated. It should be noted that the present invention is not limited to the dosages recited herein.

Those of ordinary skill in the art will readily optimize effective dosages and administration regimens as determined by good medical practice and the clinical condition of the individual recipient.

### Treatment Methods of the Invention

The invention provides methods of mobilizing hematopoietic stem cells in a donor comprising administering an effective amount of a composition comprising a multi-PEGylated G-CSF preparation to the donor. In one aspect, the invention provides methods of mobilizing hematopoietic stem cells in a donor comprising administering an effective amount of a pharmaceutical composition comprising an SD/03 preparation to the donor. As discussed above, compositions of the invention may comprise another therapeutic agent such as SCF, a chemokine antagonist or a VCAM antagonist. The methods may further include the step of isolating hematopoietic stem cells from the donor. Methods for isolating hematopoietic stem cells from a donor are routine in the art.

In another aspect, the invention contemplates a method of treating a patient in need of an allogeneic hematopoietic stem cell transplant by administering to the patient hematopoietic stem cells mobilized in a donor treated with a multi-PEGylated G-CSF preparation of the invention. In one embodiment, a method of treating a patient in need of an allogeneic hematopoietic stem cell transplant by administering to the patient hematopoietic stem cells mobilized in a donor treated with an SD/03 pharmaceutical composition.

In yet another aspect, the invention provides a method of increasing CTL function in a patient undergoing a hematopoietic stem cell transplant, comprising administering to the patient hematopoietic stem cells mobilized in a donor treated with a multi-PEGylated G-CSF composition, such as an SD/03 pharmaceutical composition.

In another aspect, the invention provides a method of increasing GVT effects in a patient undergoing a hematopoietic stem cell transplant, comprising administering to the patient hematopoietic stern cells mobilized in a donor treated with a multi-PEGylated G-CSF composition, such as an SD/03 pharmaceutical composition.

In yet another aspect, the invention provides a method of increasing iNKT cell-dependent cell clearance in a patient undergoing a hematopoietic stem cell transplant, comprising administering to the patient hematopoietic stem cells mobilized in a donor treated with a multi-PEGylated G-CSF composition, such as an SD/03 pharmaceutical composition.

A patient “in need of” a hematopoietic stem cell transplant may be a patient suffering from a disease or disorder including, but not limited to, diseases of the blood or bone marrow, and cancer. Examples include multiple myeloma, leukemia, inborn defects such as severe combined immunodeficiency or congenital neutropenia with defective stem cells, aplastic anemia, sickle-cell disease, myelodysplastic syndrome, neuroblastoma, lymphoma, Ewing's Sarcoma, Desmoplastic small round cell tumor, Hodgkin's disease, non-Hodgkin's lymphoma (NHL), renal cell carcinoma, germ cell tumor, breast cancer and, generally, neoplastic conditions of organs including both solid and liquid tissues.

“Increasing” effects or clearance is contemplated to be an increase due to the administration of a preparation of the invention such as SD/03, alone or in combination with other therapeutics, relative to the effects or clearance seen upon administration of a mono-PEGylated G-CSF preparation such as SD/01, peg-filgrastim prepared according to methods described in WO 96011953 published Apr. 25, 1996. The increase may be measured in terms of quantitative measurements of effector cells by phenotype or functional assay, or in terms of the anti-tumor or immunomodulatory activity of those effector cells.

In another aspect, patients other than those in need of a hematopoietic SCT may be treated by administering a multi-PEGylated G-CSF composition of the invention including an SD/03 pharmaceutical composition. Those patients benefit from the improved anti-tumor and/or immunomodulatory effects of cells arising from stimulation with SD/03. In some embodiments, the patient may be a patient with a solid organ malignancy, a chemotherapy patient, or a patient with an infectious disease.

### BRIEF DESCRIPTION OF THE DRAWING

FIG. 1A shows the expansion of myeloid cells (monocytes and granulocytes) was significantly greater in recipients of SD/03 versus control.

Representative plots of lineage c-kit⁺sca-1⁺ cells in the spleen six days after mobilization with SD/01 or SD/03 are shown in FIGS. 1B.

The percentage and absolute numbers of cells in spleen following SD/01 or SD/03 mobilization are shown in FIG. 1C.

Survival curves set out in FIG. 1D reveal that both SD/01 and SD/03 provided significant protection from GVHD.

FIG. 1E shows mobilization with SD/03 resulted in significantly greater CTL activity after SCT than SD/01.

FIG. 2A shows overall survival of recipients by Kaplan-Meier analysis.

FIG. 2B shows leukemic relapse in the recipients shown in FIG. 2A by Kaplan-Meier analysis.

FIG. 2C shows luminescence (photons/second/cm²/sr) over time as a determinant of leukemia burden in the recipients shown in FIG. 2A

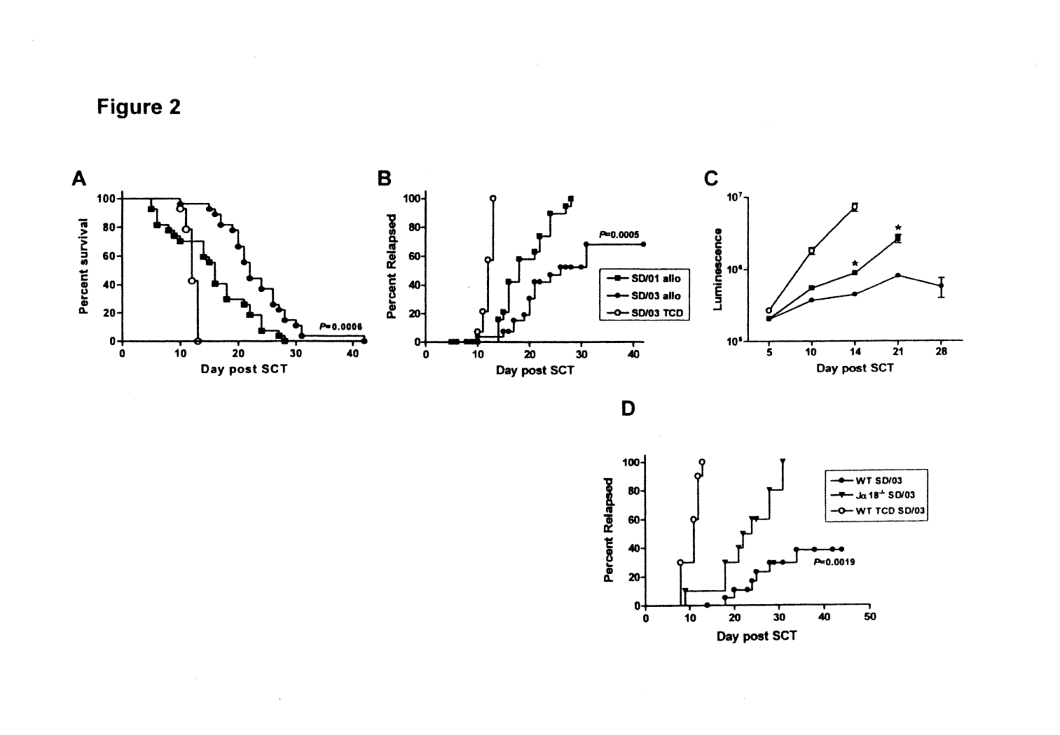

Results obtained by Kaplan-Meier analysis are shown in FIG. 2D where recipients of T cell-depleted grafts died by day 12 of leukemia while over 60% of recipients of SD/03 mobilized T cell-replete grafts survived. In contrast, recipients of SD/03 mobilized Jal8^{-/-} grafts all developed progressive leukemia with a median survival of only 23 days.

### DETAILED DESCRIPTION OF THE INVENTION

The present invention is described with reference to the following examples which are offered to illustrate the invention, but are not to be construed as limiting the scope thereof. Example 1 sets out a method to make SD/03. Example 2 describes the mobilization of hematopoietic stem cells with SD/03. The effect of mobilization with SD/03 in donors on GVHD in recipients is described in Example 3. Example 4 reports the effect of mobilization with SD/03 in donors on CTL generation. Example 5 describes the effect of mobilization with SD/03 in donors on recipient survival and iNKT-dependent GVL activity.

### Example 1

### SD/03 Preparation

SD/03 can be produced from Filgrastim, the active ingredient in NEUPOGEN® (Amgen Inc., Thousand Oaks, Calif.). SD/03 is a sustained duration form of Filgrastim produced by covalent attachment of 20 kD polyethylene glycol (PEG) molecules to the Filgrastim polypeptide chain.

The process includes the PEGylation reaction of 20 kD PEG-aldehyde and Filgrastim, and purification steps including an ion exchange chromatography column, an ultrafiltration and diafiltration step, formulation and final filtration.

The PEGylation reaction is carried out in mildly acidic to alkaline conditions (pH>6) and in the presence of sodium cyanoborohydride at ambient temperatures. Higher and lower reaction temperatures can be successfully used with the primary impact to the relative reaction rate. The PEG-aldehyde to protein ratio used was between 3 and 6 moles of PEG per mole of Filgrastim and the reaction was carried out for a duration of 8 to 24 hours. Higher and lower PEG ratios and reaction durations can be used successfully with the primary impact on the extent of PEGylation. Under the above conditions the PEG aldehyde forms covalent linkages to Filgrastim.

Subsequent to the reaction, the pH was adjusted to mildly acidic conditions (pH 4.5), filtered and loaded on an SP Sepharose High Performance, or equivalent, cation exchange column. The column was pre-equilibrated with 20 mM sodium acetate, 5% glycerol, pH 4.5. The column is then washed with 20 mM sodium acetate, 5% glycerol, pH 4.5 and eluted with a linear salt gradient from 0 mM to 150 mM sodium chloride 5% glycerol, 20 mM sodium acetate, pH 4.5 over 7.5 column volumes. Fractions were collected and analyzed using cation exchange high performance liquid chromatography. Fractions with unreacted Filgrastim and mono-PEGylated species were discarded and the remaining higher PEGylated species were combined to form Filgrastim SD/03.

The resulting mixture was diafiltered using a 10 kD NMWL (or equivalent) membrane against a solution of 10 mM sodium acetate, 5% w/v sorbitol, pH 4.0. Membranes with higher or lower NMWL can be successfully used with the primary impact to duration of diafiltration and/or filtration yield. The resulting diafiltered PEGylated polypeptide was filtered through 0.45 micron pore size filter and the pH was further adjusted to 4.0 as necessary.

### Example 2

### Mobilization of Hematopoietic Stem Cells with SD/03

The effect of administration of SD/03 on BMSC mobilization in mice was compared to administration of SD/01.

SD/01 or SD/03 was administered to donor B6 mice at a clinically achievable dose (3 μg/dose, equivalent to 150 μg/kg). Mice were housed in sterilized micro-isolator cages and received acidified autoclaved water (pH 2.5) post-transplantation. Six days later spleens were phenotyped and total numbers of each cell lineage elucidated per spleen (n=5 or 6 per group).

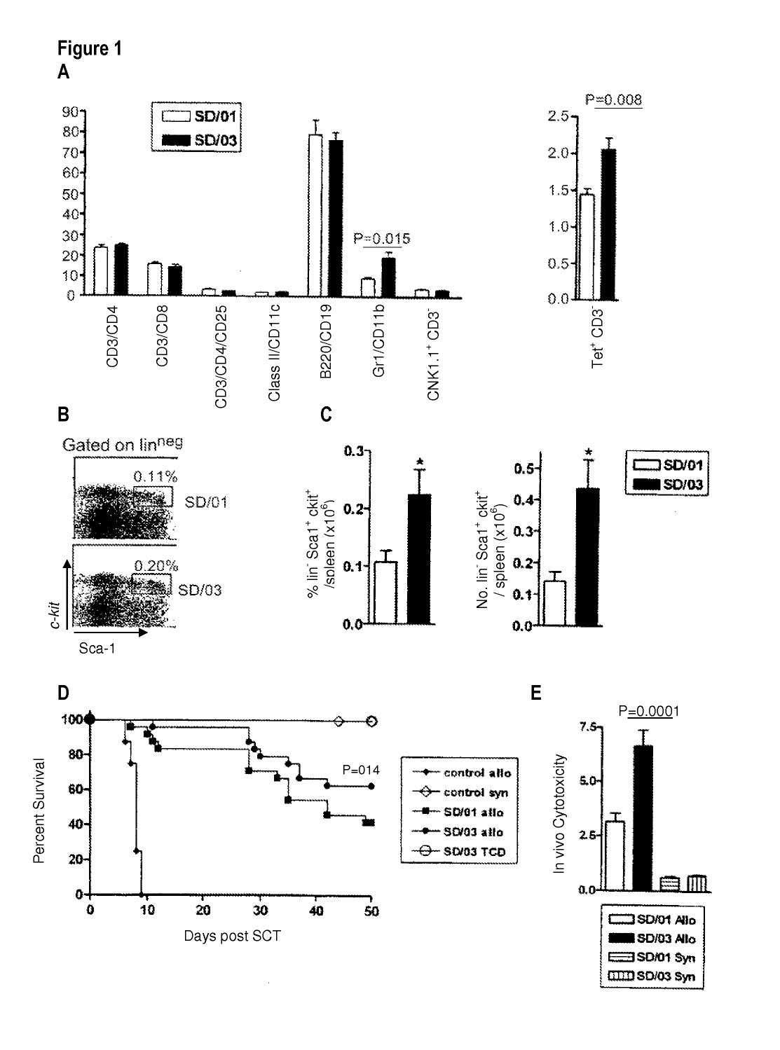

As demonstrated in FIG. 1A, the expansion of myeloid cells (monocytes and granulocytes) was significantly greater in recipients of SD/03 (note that granulocytes are <4×10⁶ per spleen in control animals). Numbers of other lineage positive cells were similar.

In order to determine relative stem cell mobilization, lineage negative, c-kit³⁰ sca-1⁺ stem cells were quantified within the spleen. Flow cytometry was undertaken as described in Morris et al., J. Clin. Invest., 115: 3093-3103 (2005), while the determination of lineage negative (Mac-1, Gr-1, CD4, CD8 and TER119), c-kit and Sca-1 positives cells was undertaken as described in Okada et al., Blood, 80: 3044-3050 (1992). Representative plots of lineage c-kit⁺sca-1⁺ cells in the spleen six days after mobilization with SD/01 or SD/03 are shown in FIGS. 1B. The percentage and absolute numbers of cells in spleen following SD/01 or SD/03 mobilization (n=4 per group) are shown in FIG. 1C. SD/03 significantly increased the frequency and number of stem cells while proportions and numbers in the marrow were equivalent, consistent with an enhanced ability for SD/03 to mobilize stem cells.

### Example 3

### Effect of Mobilization with SD/03 on GVHD

Splenic grafts were transplanted into MHC disparate, lethally irradiated B₆D₂F₁ recipients as previously described in Morris et al. (2005), supra; Morris et al. (2004), supra and MacDonald et al., Blood, 101: 2033-2042 (2003). B₆D₂F₁ (H-2^{b/d}, CD45.2⁺) mice were purchased from the Animal Resources Centre (Perth, Western Australia, Australia).

Briefly, on day-1, B₆D₂F₁ mice received TBI (1100 cGy) split into two doses separated by three hours to minimize gastrointestinal toxicity. The mice were transplanted at day 0 with 10⁷ splenocytes from B6 donors mobilized by SD/01 (SD/01 allo, n=24) or SD/03 (SD/03 allo, n=24), equilibrated to deliver equal T cell doses. Control B₆D₂F₁ recipients received transplants from saline treated allogeneic B6 donors (control allo, n=8) or syngeneic B₆D₂F₁ donors (control syn, n=9). Additional control recipients were transplanted with T cell depleted (TCD) allogeneic grafts from SD/03 mobilized B6 donors (SD/03 TCD, n=4). Transplanted mice were monitored daily and those with GVHD clinical scores of 6 were sacrificed and the date of death registered as the next day in accordance with institutional animal ethics committee guidelines. The degree of systemic GVHD was assessed by scoring as described (maximum index=l0) in Cooke et al., Blood, 88: 3230-3239 (1996). Results were pooled from three experiments and survival curves were plotted by using Kaplan-Meier extimates and compared by log-rank analysis. P<0.05 was considered statistically significant.

Both SD/01 and SD/03 provided significant protection from GVHD. Significant differences between SD/01 and SD/03 were not apparent (FIG. 1D), but in each experiment SD/03 appeared marginally superior.

### Example 4

### Effect of Mobilization with SD/03 on CTL Generation

The activation of donor iNKT cells by SD/01 with subsequent enhancement of donor CTL function is demonstrated in Morris et al. (2005), supra. The effect of SD/03 on CTL generation in SCT recipients was determined as described below.

Briefly, irradiated allogeneic B₆D₂F₁ recipients were transplanted with allogeneic B6 or syngeneic B₆D₂, F₁ splenocytes mobilized with SD-01 (SD/01 allo, n=15, SD/01 syn, n=3) or SD-03 (SD/03 allo, n=16, SD/03 syn, n=6). At day +12, the in vivo cytotoxicity index was determined as previously described in Morris et al. (2005), supra and Banovic et at, Blood, 106: 2206-2214 (2005) by determining the clearance of adoptively transferred host versus donor splenocytes. Data are represented as mean ±SE from 3 experiments.

As shown in FIG. 1E, mobilization with SD/03 resulted in significantly greater CTL activity after SCT than SD/01.

### Example 5

### Effect of Mobilization with SD/03 on Survival and iNKT-Dependent GVL Activity

In order to study the effect of mobilization with SD/03 on GVL effects, a clinically relevant MHC-matched (B10.D2→DBA/2) SCT model was utilized in which recipients also received host-type luciferase expressing leukemia (P815) at the time of transplant.

Irradiated (1000 cGy) DBA/2 recipients were transplanted with allogeneic B10.D2 splenocytes (2×10⁷ cells per mouse) mobilized with SD/01 or SD/03 splenocytes (n=27 each) equilibrated to deliver equal T cell doses. DBA/2 (H-2^{d}) mice were purchased from the Animal Resources Centre (Perth, Western Australia, Australia). Non-GVHD controls received SD/03 mobilized splenocytes that were T cell depleted (n=15). Leukemia was induced in all recipients by co-injection of 5×10³ host-type luciferase-expressing P815 cells on day 0. The mastocytoma cell line, P815 (H-2^{d}, CD45.2⁺), was derived from DBA-2 mice. Data were pooled from three experiments. Survival and clinical scores were monitored daily and the cause of death (determined by post-mortem examination) established as GVHD or leukemia. In vivo imaging was performed using the IVIS Imaging System (Xenogen, Calif.) and light emission is presented as photons/second/cm²sr.

FIG. 2A shows overall survival of recipients by Kaplan-Meier analysis. FIG. 2B shows leukemic relapse in the recipients shown in FIG. 2A by Kaplan-Meier analysis. FIG. 2C shows luminescence (photons/second/cm²/sr) over time as a determinant of leukemia burden in the recipients shown in FIG. 2A. Results are mean ±SE from 3 experiments, *P<0.05, SD/01 allo versus SD/03 allo. All TCD recipients developed leukemia on day 10 and required sacrifice prior to day 14.

The recipients of SD/03 mobilized grafts demonstrated significantly improved overall survival (FIG. 2A) relative to recipients of SD/01 mobilized grafts due to enhanced leukemia eradication (FIG. 2B) that was confirmed by biophotonic imaging post SCT (FIG. 2C).

In order to confirm that this result was indeed related to effects on iNKT cells, wild-type (WT) and iNKT deficient (Jal8^{-/-}) B6 donors were mobilized with SD/03 and grafts transplanted into irradiated B₆D₂F₁ recipients in the presence of host-type leukemia and GVL monitored thereafter. Jαl8^{-/-} B6(H-2^{b}, CD45.2⁺) mice were supplied by Mark Smyth (Peter MacCullum Cancer Centre, Melbourne, Australia). More specifically, B₆D₂F₁ recipients were transplanted with SD/03 mobilized splenocytes from allogeneic wild-type (WT SD/03, n=20), NKT deficient Jαl8^{-/-} (Jαl8^{-/-}, SD/03, n=20) or T cell-depleted WT (WT TCD SD/03, n=10) B6 donors in conjunction with 5×10⁴ host-type P815 leukemia cells. Results obtained by Kaplan-Meier analysis are shown in FIG. 2D.

As shown in FIG. 2D, recipients of T cell-depleted grafts died by day 12 of leukemia while over 60% of recipients of SD/03 mobilized T cell-replete grafts survived. In contrast, recipients of SD/03 mobilized Jαl8^{-/-} grafts all developed progressive leukemia with a median survival of only 23 days.

The ability of PEGylated G-CSF to modulate the immune system to greater levels than standard G-CSF is likely to be the result of a different exposure profile. This appears to allow the molecule to invoke effects in cell subsets that are otherwise not demonstrable following standard G-CSF administration, namely iNKT cells [reviewed in Morris et al. (2006), supra]. The activation of donor CD4^{neg}CD8^{neg} iNKT cells thereafter improves CTL printing via effects on host APC. The additional increase in biological activity by multiple-pegylation is likely to be imparted by optimization of the same mechanisms. However it is important to note that these effects cannot be reproduced by administering escalating doses of standard G-CSF. See Morris et al. (2005), supra.

Mobilizing stem cells with multi-PEGylated versions of G-CSF may thus represent an additional therapeutic alternative for patients, one that may be particularly useful in the allogeneic SCT setting to further separate GVHD and GVL.

Variations on the subject matter of the following claims will be apparent to those of skill in the art upon review of the present disclosure, and such variations are within the scope of the invention contemplated.

1. (canceled)

2. An SD/03 preparation made by a) attaching 20 kDa PEG-aldehyde moieties to Filgastrim polypeptide by a reductive alkylation reaction, wherein the reaction is carried out for 8 to 24 hours at ambient temperature and at a pH from pH 6 to pH 8.5 in the presence of sodium cyanoborohydride, and b) separating the multi-PEGylated polypeptide from unreacted and mono-PEGylated polypeptide.

3. (canceled)

4. A method of mobilizing hematopoietic stem cells of a donor comprising administering an effective amount of a composition comprising an SD/03 preparation to the donor.

5. The method of claim 4 wherein the composition comprises another therapeutic agent, wherein the therapeutic agent is stem cell factor, a chemokine antagonist or a VCAM antagonist.

6-8. (canceled)

9. The method of claim 4 further comprising the step of isolating hematopoietic stem cells from the donor.

10. A method of treating a patient in need of an allogeneic hematopoietic stem cell transplant, comprising administering to the patient hematopoietic stem cells mobilized in a donor treated with an SD/03 pharmaceutical composition.

11-13. (canceled)

14. The method of claim 10 wherein the patient has leukemia.
